(19) United States
(12) Patent Application Publication
(10) Pub. No.: US 2016/0136384 A1
(43) Pub. Date: May 19, 2016

(54) SYSTEM, METHOD AND KIT FOR REMINISCENCE THERAPY FOR PEOPLE WITH DEMENTIA

(71) Applicant: TREASURE VOX LLC, Hartford, CT (US)

(72) Inventor: Janet C. COVER, Hartford, CT (US)

(73) Assignee: TREASURE VOX LLC, Hartford, CT (US)

(21) Appl. No.: 14/547,130

(22) Filed: Nov. 18, 2014

(51) Int. Cl. A61M 21/02

(52) U.S. Cl. CPC A61M 21/02; A61M 2021/0027

(57) ABSTRACT

A system, method and kit for reminiscence therapy on an individual and group setting to treat depression and other disorders based on timing and application of reminiscence therapy using the compositions of one or more audio and visual elements configured to constructively interact with the individual's autobiographical memory components to stimulate memory components of the life of the individual with dementia.

This patent application claims priority to U.S. Provisional Patent Application Ser. No. 61/905,693 entitled “A DIGITAL PHOTO ALBUM WITH A BUILT-IN VOICE RECORDER” filed on Nov. 18, 2013, and the entire disclosure is hereby incorporated by reference herein.

### FIELD OF THE INVENTION

The patent relates to reminiscence group therapy to improve quality of life of people with dementia and to improve relationships with carers and, more particularly, to a system, method and kit for preparing and applying compositions of one or more audio, visual, and memoralbilia objects for use in reminiscence therapy into a portable multi-dimensional device, and the use of such compositions to treat various forms of dementia.

### BACKGROUND OF THE INVENTION

Neurological system disorders, such as persons living with dementia, represent potentially incapacitating diseases affecting a growing number of people in the population. Current approaches to the treatment of dementia involve pharmacological interventions using medications. However, problems can occur as medication may have negative side-effects in a person due to physiological and pharmacological factors. Moreover, there is a factor of the cost of care and medication in pharmacological interventions for the treatment of dementia. As a result, there is a long-felt need for therapeutic interventions intended to benefit people with dementia and their family carers where medication may be ineffective or have negative side-effects.

In recent years, research has been conducted into the use of reminiscence therapy as a psycho/social approach to working with people with dementia. Typically, in a one on one setting, together with a reminiscence (narrative therapist) and an older adult, uses “memory triggers” (photographs, recordings, artefacts etc.) to promote personal and shared memories. A recent development has been to include family carers in reminiscence groups alongside their relatives with dementia. Descriptive evaluations suggest that this joint approach (described as ‘Remembering Yesterday Caring Today’—RYCT) may improve the relationship between carer and person with dementia, benefiting both.

As a result, there is increasing recognition that psycho/social interventions may have comparable value and may be preferable in some contexts, e.g. where medication may be ineffective or have negative side-effects. Reminiscence individual or group therapy, run by professionals and volunteers, which use photographs, recordings and other objects to trigger personal memories are one of many of the popular therapeutic approaches to working with people with dementia. Problems occur, however, with set-up logistics, cost and time in assembling the photographs, recordings and other objects as certain family members are in different geographic locations and the collecting and presenting such assembly of such objects and things can be one-dimensional.

As a result, there is a long-felt need for an improved. cost-effective system and method for interventions using reminiscent therapy to benefit people with dementia and their family and professional carers. The present invention satisfies such need for a system, method and kit for persons with dementia, and their carers including family, providing an efficient, cost effective reminiscence therapy. The present invention efficiently and cost effectively forms assembles from the person's group photographs, recordings and other objects useful to trigger personal memories and captures the stories that benefits such person(s) with dementia and their carers.

The present invention satisfies a long-felt need for a solution in a portable multi-dimensional apparatus and system for a digital photo album with a built-in voice recorder product and ancillary services with applications that include providing autobiographical memories in Alzheimer's and dementia patients. The present invention improves relationships between people with dementia and their carers and improves overall wellbeing in the patient. The present invention provides an equally effective alternative to medicating such person with dementia.

### SUMMARY OF THE INVENTION

It is an object of the present invention to provide an apparatus, system, method and kit for reminiscence therapeutic application to improve quality of life of an individual with dementia by stimulating an elevated mood and reducing levels of depression, anxiety, cognitive dysfunction, dysfunctional psycho-physiological regulation capacity, and other disorders.

A method for reminiscence therapeutic application to improve quality of life of an individual with dementia, comprising the steps of: (a) composing autobiographical memory components of the life of the individual with dementia in a portable computing device; (b) incorporating one or more visual elements in such autobiographical memory components, whereby said visual elements being configured to stimulate memory components of the life of the individual with dementia; (c) incorporating one or more audio elements in such autobiographical memory components, whereby said visual elements being configured to stimulate memory components of the life of the individual with dementia; (d) creating a composition of said one or more audio and/or visual elements of said autobiographical memory components of the life of the individual with dementia; and (e) presenting said composition to the individual with dementia by playing back said one or more audio and/or displaying said one or more visual elements so as to stimulate improvement in the mood of the individual with dementia.

It is an object of the present invention to provide a device comprises a readable medium for storing an computer program, wherein said computer program accepts input(s) for receiving autobiographical memory components of the life of the individual with dementia with the autobiographical memory components comprising: (1) one or more visual elements in such autobiographical memory components; (2) one or more audio elements in such autobiographical memory components; and (3) a composition of these one or more audio and visual elements configured to constructively interact with the individuals autobiographical memory components configured to stimulate memory components of the life of the individual with dementia. The device includes a playback device for reading the readable medium and playing back the audio and visual elements to the individual.

It is an object of the present invention to provide a device and system for therapeutically treatment of an individual with dementia by using the device to play said composition to the individual so as to stimulate improvement in the mood of the individual, evaluating an individual's psycho-physiological state to determine psycho-physiological needs and dysfunctions, and selecting a treatment plan and schedule to treat psycho-physiological needs and dysfunctions for the individual with dementia.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations.

FIG. 1 illustrates a schematic view of apparatus, system and method of the present invention;

FIG. 2 illustrates a schematic view of apparatus, system and kit of the present invention;

FIG. 3 illustrates a schematic flowchart view with screen shot views of setup of the apparatus, system and method of the present invention;

FIG. 4 illustrates a schematic flowchart view with screen shot views of adding memory content of a photograph, video or the like using the apparatus, system and method of the present invention;

FIG. 5 illustrates a schematic flowchart with screen shot views of adding memory content of a recording using the apparatus, system and method of the present invention;

FIG. 6 illustrates a schematic flowchart with screen shot views of playback of memory content of a recordings, photographs, video and the like using the apparatus, system and method of the present invention; and

FIG. 7 illustrates a schematic flowchart with screen shot views of administrative management of memory content of a recordings, photographs, video and the like using the apparatus, system and method of the present invention;

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “Treasure Vox” refers to a portable multi-dimensional apparatus and system for digital photo album with a built-in voice recorder product and ancillary services. Treasure Vox® also source identifiers for digital photo album with a built-in voice recorder product and ancillary services. The Treasure Vox® product can be a software application designed for use with a Tablet. The product and service allows a user create content memory items using the Tablet to (1) download content of photographs, recordings, and/or video from a connection (e.g. Network, Internet, Telephone Provider, Bluetooth, etc.), (2) to use photographs, recordings, and/or video that exist on the tablet, or (3) to create content photographs, recordings, and/or video using the Tablet. In a playback mode, the computer-implemented program displays the images one at a time on the screen of the Tablet. While the image is displayed in the viewer window of the application, the user is given an option to use the built-in audio record feature by depressing or touching a record button. Once the audio recording is finished, the user may playback the message associated with each photograph. This can be repeated overtime to allow for the digital photo album to grow and be updated.

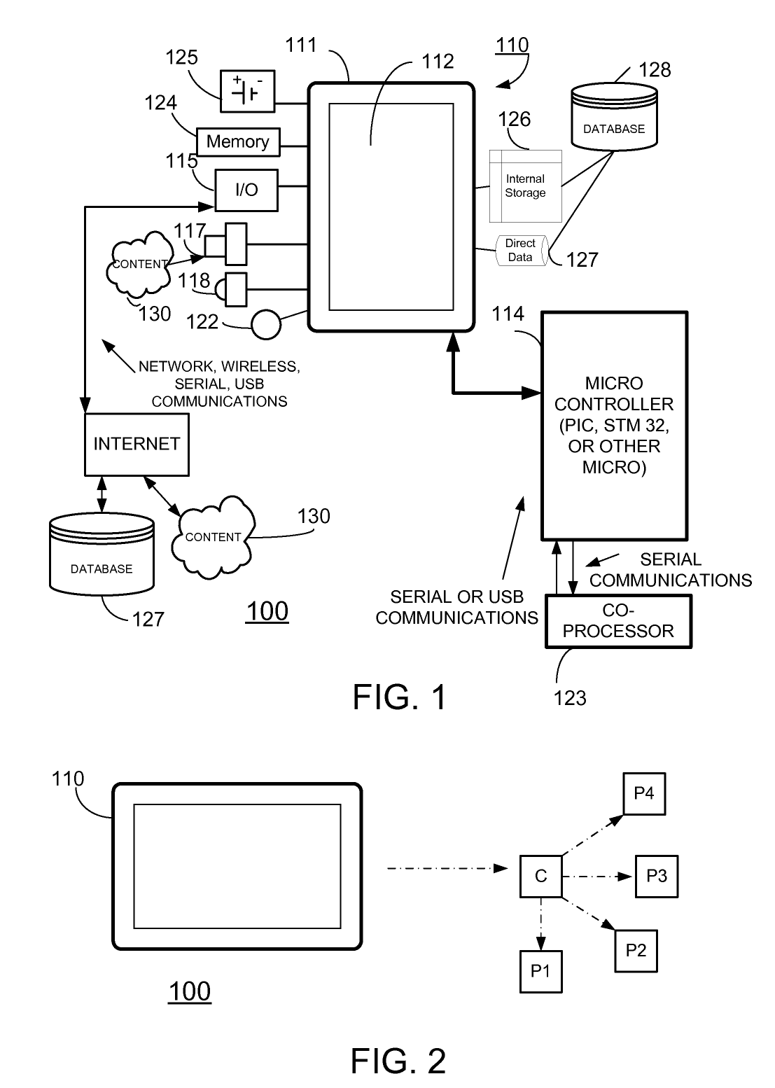

Referring to FIGS. 1 through 7, a reminiscence therapy apparatus, system, method and kit 100 includes a portable computing tablet device 110 (e.g. a handheld tablet, Smartphone etc.) which acts as a tangible interface for allowing a user to interact with digital content displayed on its display or screen 111. The screen may have a touch enabled screen 112. The reminiscence therapy apparatus, system, method and kit 100 is described in an embodiment of a tablet 110 used to compose autobiographical memory components of the life of the individual with dementia.

The device 110 can be used in a hospital environment (e.g. in a care unit or the like) and when the person treatment therapy, (e.g. as may be given to a person to interact individually with a family or carer, and with others is undergoing in a group). The device 110 has been found to be effective in improvements in autobiographical memory, life and mood, communication and interaction, caregiver stress relief and better relationships with family, caregivers and other elders living in a community settings in the treatment of dementia, and accordingly has been found to improve the feeling of happiness and well-being and to reduce the pain and anxiety felt by the patient.

The tablet device 110 and digital content are designed to provide an immersive experience for the user, and the device 110 allows the user to interact with the content in an intuitive and immediate fashion, without complicated controls or the like, and without any great need for assistance from clinical staff. The tablet device 110 the display screen 111 may take any suitable form, and may be for example an LCD screen, e.g. a TFT screen or the like. It may include a stereoscopic screen, so that the three-dimensional scenes displayed appear to be in physical three-dimensional space. The display screen 111 may also incorporate a touch screen 112, so as to provide a further way in which a patient may interact with the device. The touch screen 112 may allow a patient to select an object in a scene, activate a function or allow for movement within the scene.

The tablet device 110 houses a motion sensor 113 may take any suitable form, for example, incorporate any suitable inertial measurement unit, which may be a number of motion sensors, such as gyroscopes and/or accelerometers. The device may sense movement in three-dimensions, and may sense translations in three-axes as well as rotations about those axes. It may determine pitch, roll and yaw, and accelerations in three perpendicular axes. The motion sensor 113 monitors the position and motion of the tablet device 110. The device 110 includes a controller 114 that determines the movement and position of the tablet device 110 from the output of the motion sensor 113 and that provides appropriate content to the screen 112 based on the determined motion.

The controller 114 may take any suitable form, and may be a suitably programmed microprocessor 123 with associated memory 124 and video capabilities, and may run an appropriate operating system and applications. It may be a single unit, or may comprise a number of separate control units, e.g. a separate video processor, a separate motion processor and the like. The controller 114 may also include suitable input/output modules 115, which could include a wireless transceiver, a USB connection or the like. As a result, the device 110 may download new content or the like from members of the individual's group (e.g. family, carers, reminiscent group therapy participants, doctors, study content, etc.), e.g. from a data carrier, a database, a network server or the like, and/or connect to external processing devices. The controller 114 may be a microprocessor 123 or any other suitable processing element. It need not be a single unit, but could include a number of processing units, e.g. a separate graphics processor, motion processor or the like. The device 110 may include memory 124 accessible by the controller 114 for storing digital content, applications, operating systems and the like. The device 110 may be powered by rechargeable batteries 125.

The tablet device 110 also includes buttons 116, either in the device or in the form of a software button displayed on a touch enabled screen, for allowing various functionality. A button 116 may for example reset the displayed content to the start position, or could allow for zooming into or out of the displayed scene. The functions of the buttons may change depending on the content being displayed and the story or game or such like that is being provided. Once content is selected using a button 116, an appropriate “memory trigger” (photographs, recordings, artifacts etc.) appears on the screen 111 allowing the user to interact with the scene, e.g. to move through the displayed environment, to interact with entities in that environment, and to tell the person's story associated with such memory.

The controller 114 may be configured to allow further forms of interaction with a camera 117 for taking visual images (e.g. photographs, video, etc.), light or LED 118 (i.e. for a flash or to illuminate objects and things), microphone 119 for the input/output of sound, speaker 120 for the output of sound, vibratory motor 121 for the output of vibration, and a variety of other sensors such as any necessary physiological sensors 122 (i.e. a physiological sensor measuring heart rate, blood pressure, brain activity, muscle tension, skin conductivity, respiratory rates, oxygen saturation, general biometrics, or the like).

The movement of the device 110 could provide such functionality through various specific tablet device movements that may be associated with a function. For example, translational movement of the tablet device in a forward or backwards direction could be a trigger for a zoom in or a zoom out, and a vigorous shake of the device could provide a reset. The device 110 also includes buttons 116, in the form of touch pads, for allowing various functionality. A button may for example reset the displayed content to the start position, or could allow for zooming into or out of the displayed scene. The functions of the buttons may change depending on the content being displayed and the story or game or such like that is being provided.

The tablet device 110 is configured with the camera 117 and LED 118 so as to capture an image (e.g. photo, video, etc.) and the LED 118 lights the images. The camera may be used to capture digital images that may then be displayed on the screen, and may provide an augmented reality device, e.g. a device which superimposes or otherwise blends virtual features onto a real world image. The captured image may then be processed using appropriate image processing software and stored in the memory 124 of the tablet 110 or on the network. The controller 114 can take appropriate action dependent on a particular composition and to allow for selection of video functions such as, for example, a fast forward or rewind of a video or the like. The camera 117 may provide a number of different functions, and may significantly increase the interaction and engagement of the patient with the device. The camera 117 may be a still image camera or a video camera, and may for example be a web-camera. It may be mounted in any suitable location on the device. In one preferred form, the camera is provided on the front of the device facing the user. This allows the user to capture themselves on the screen easily.

As well as inputting and outputting content to a display 111, the tablet device 110 is configured to input and output an audio signal, such as a narration, music, sound effects or the like, e.g. through a microphone 119 or a speaker 120, e.g. using a surround sound device, and may provide haptic feedback, e.g. through the vibratory motor 121, that will vibrate the device 110 under certain conditions, e.g. when a particular event occurs in the composition of said one or more audio and/or visual elements of the autobiographical memory components of the life of the individual with dementia. The camera 117 may form part of the motion sensing system 113 for the device 110 to capture images in the camera's 117 field of view.

The physiological sensors 122 can provide ECG, EEG or EMG information and data the individual's reaction to the use of the reminiscence therapy apparatus, system, method and kit 100 and reaction with the content based on a control of their physiological condition. According to embodiments of the present invention, the device 110 may encourage a user to control the condition, e.g. by relating the condition to achieving a result or the like, or by visualizing the condition to the user. Moreover, the tablet device 110 and its controller 114 may be configured with a microprocessor 123, memory 124, and rechargeable batteries 125 so as to provide a computing device with a computer implemented software application designed for use with a tablet 110.

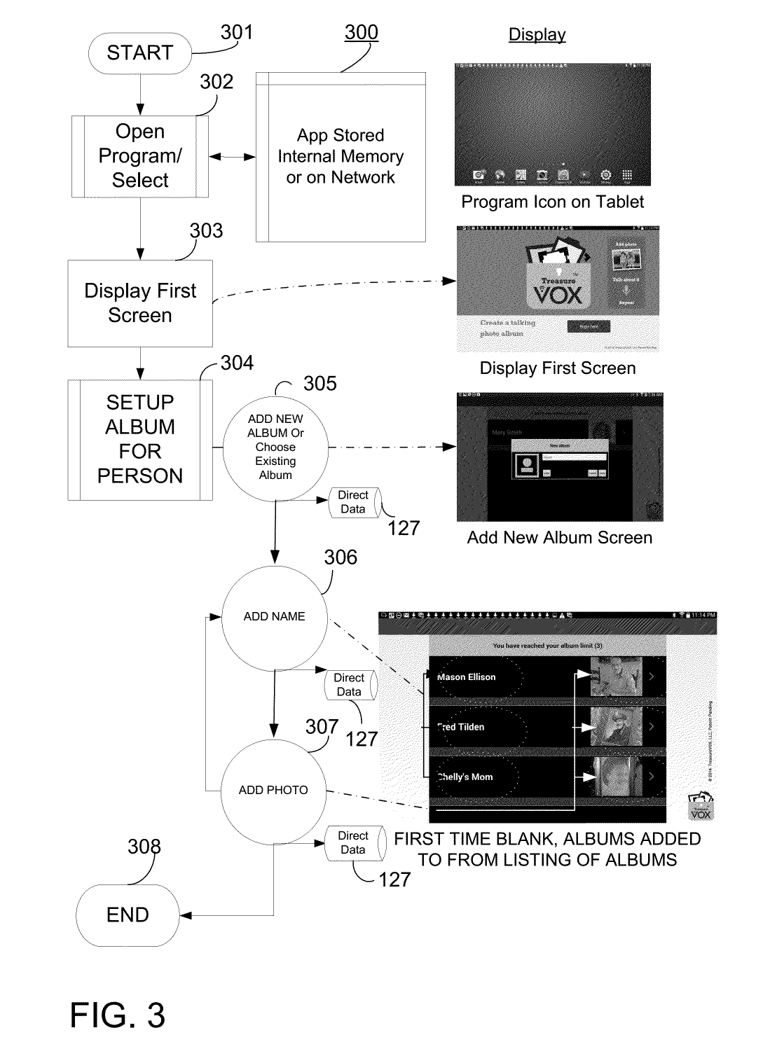

The reminiscence therapy apparatus, system, method and kit 100 can be a computer implemented process as a software application designed for use with a tablet 110. As is shown in FIGS. 3 through 7, a computer implemented process 300 can be configured to store the visual and audio content to memory 124, internal storage 126, receive direct data 127 input from the user and with data storage 128 on the tablet 110 or backed-up to a server on a network (FIG. 1). A catalog or listing of the internal storage 129 may be assembled for such received direct data 127 and audio and visual content data storage 128 on the internal storage 126 of the device by software application designed for use with a tablet 110.

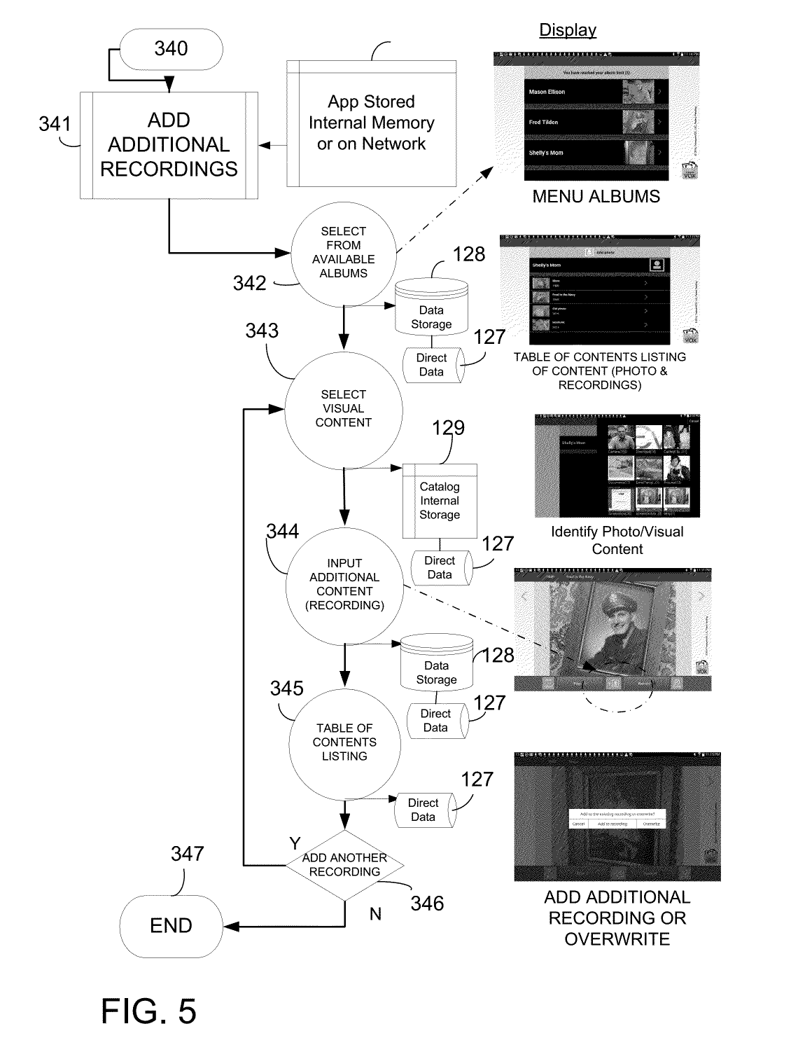

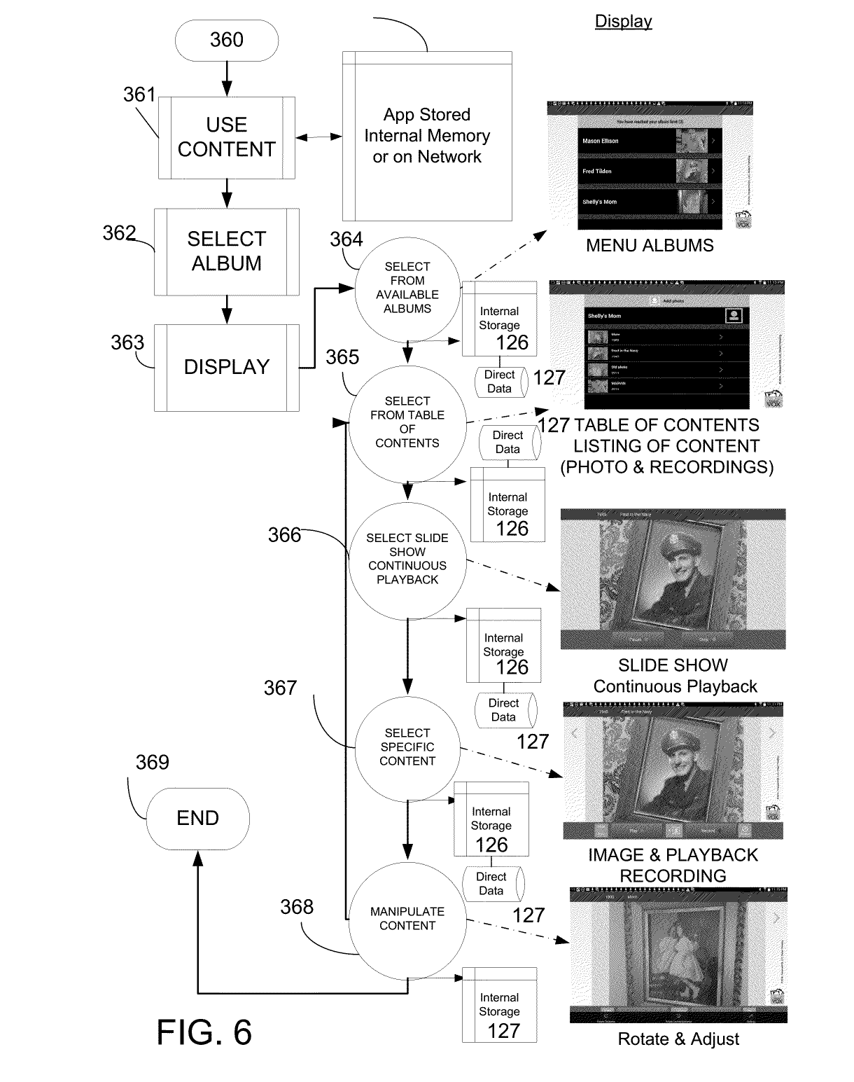

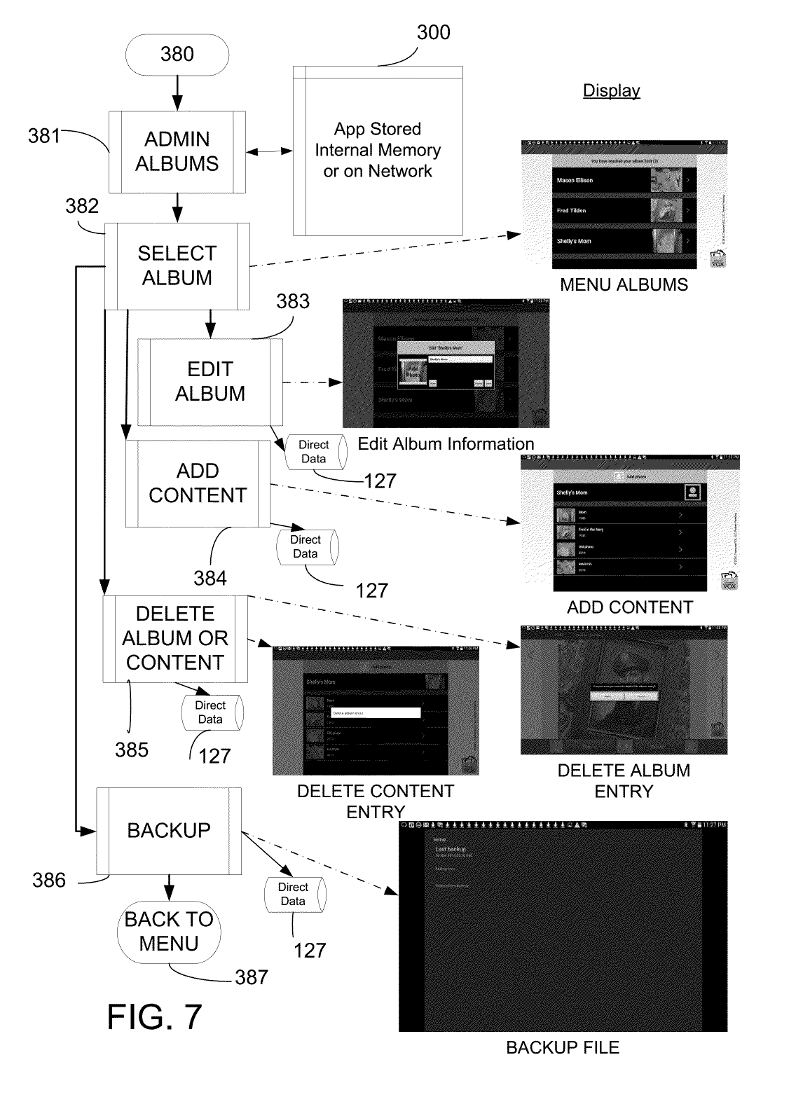

The computer implemented process 300 can be formed as a software application, commonly referred to as an APP, for such as a tablet, smart phone, and the like, e.g. Apple or Android tablet devices and operating systems. The App uses the touch screen of a device 110, for example, an android phone with an android App 300 provides a way for recording and playing back memos on a per user basis. The App 300 can have multiple user profiles, can allow allows for the input of memories or memos, and advantageously, as shown in FIG. 5, one or more audio contents (e.g. one or more voice recordings or memo) can be associated with visual content (i.e. an image or video). Each memo will be viewable in the device 110 and audio can be played back. Administrative functions, as shown in FIG. 7, allow for each album to be edited, content added, content backed-up, or if each recording or album should need to be deleted. Each image is allowed to be changed and manipulated (e.g. rotated and adjusted) as shown in FIG. 6. Moreover, the visual content and photos may be stored, indexed, cataloged in a database so as to be sort-able by picture date, created date or title. Album and multiple records can be stored for each profile. In operation, the computer implemented process 300 allows a user to download photographs or use photographs that exist on the tablet 110 and to display the images one at a time on the screen 111 of the tablet. While the image is displayed in the viewer window of the application, the user is given an option to use the built-in audio record feature by depressing or touching a record button. Once the audio recording is finished, the user may playback the message associated with each photograph. Each recording with associated photograph is backed up to a web server for archival and extended services including sharing with selected family and friends and conversions to a personal movie.

Referring to FIG. 2, in the clinical setting a care-giver or carer (“C”) utilizes the device 110 with the computer implemented program 300 installed to provide reminiscent therapy to one or more patients P₁, P₂, P₃, P₄, . . . Pₙ This phase operating the App 300 allows individuals and other people living with dementia to play the composition to the individual according to a treatment schedule designed to treat the disorder. Presenting the audio visual composition to the individual with dementia by playing back said one or more audio and/or displaying said one or more visual elements stimulate improvement in the mood of the individual with dementia.

As is shown in FIG. 3, computer implemented process 300 generally displays an icon on the tablet 110 operating to launch or “start” in step 301 the computer implemented program 300. Once the icon 301 is touched, the second screen is displayed. In Step 302, when the user (i.e. patient, caregiver, carer, family member etc.) wants to access the program, touch screen 112 of the tablet 110 is used to depress the image of the associated the icon to gain entry to the application. In Step 303 a first start screen displays an overview and description of the computer implemented program 300. In Step 304 the user is directed to “Begin Here” using software button, whereby the touch screen 112 of the tablet 110 is used to depress the image of “Begin Here”. Once the initial setup is complete for the person, the user can go directly to adding new memory trigger content by “Add Photo” and “Talk About It” “Repeat” buttons on the initial screen.

In Step 305, the user utilizes the App 300 to establish a new profile for a user. Inputs are requested via a direct input (i.e. a pop-up keyboard etc.) and the user requested to enter in Step 306 a Name (e.g. first and last name) to create a unique profile account, and in Step 307 to add a Photo, which is entered as direct data 127 and stored as a unique profile account is associated with memory 124, internal storage 126, and data storage 128 of the Tablet 110. The database indexes the data to provide a catalog of a audio and visual content associated with the unique profile account. This process may be repeated to form other users and set-up other accounts. When finished, the process ends in Step 308 to take the user back to the menu. It enables a user to have ownership to their own photo/recording slideshow. When the first and last names are entered, the user selects save. The App allows advantageously an unlimited number of users the ability to add their own user profile. The number of users may include a husband and wife and immediate family members.

In Step 310, the user utilizes the computer implemented process 300 of the App to add new content. In Step 311, the process requests the user to select from available albums assembled into a menu by the process 300 by the database. In Step 312, the user selects from a profile account or listing of accounts appears in the “Profiles” screen or menu of albums. Touching or selecting the person's name opens the next screen.

In Step 313, a Table of Contents screen appears after the user selects the desired item, e.g. the bar with their name on it, and direct data 127 is entered. In Step 314, the user selects “Add photos” located below the heading in the window box, and direct data 127 is entered. In Step 314, the user is then prompted to select one photo from the tablet's photo “Gallery” that is available from internal storage 126, or to take a photo using the device 110. The gallery shows all photos available to the user. In Step 315, the user selects the image and is prompted to add a description and name, and in Step 316, to add a date to represent when it was taken and a title to the photograph, and direct data 127 is entered. The user may also wait to add the date and title since the information may be typed in the view window. After the information is saved in date/title dialog box the user is automatically moved to the photo/audio record viewer, in Step 317.

In Step 317, advantageously, input from the microphone 119 of the device 110, or a audio file, the content is augmented by the voice recorder and the photographic viewer. In Step 317, the photograph is displayed, the text line that includes the date and the title to the photo, as well as the “play” and “record” buttons to capture and to listen to the spoken stories. To the left and right of the photos are arrows that allow the user to move to the next image or back when more than one image has been downloaded into the application. A delete button serves to delete the photo and the recording. When that is depressed the user is asked if they wish to delete the photo and voice recording. To the far left of the play button is an icon that when depressed allows the person or user to add more photographs. If the user should want to add a new photo, the computer implemented process 300 of the App will take the user back to Step 314 to add content; otherwise, the process will end at step 319.

In Step 317. to activate the voice recorder the user touches the record button. A stop or “save” button appears during the recording. A message appears telling the user the recording is playing. To play back the voice or audio recording, depress the “play” button. When the play button is depressed, a “pause” and “stop” button appears.

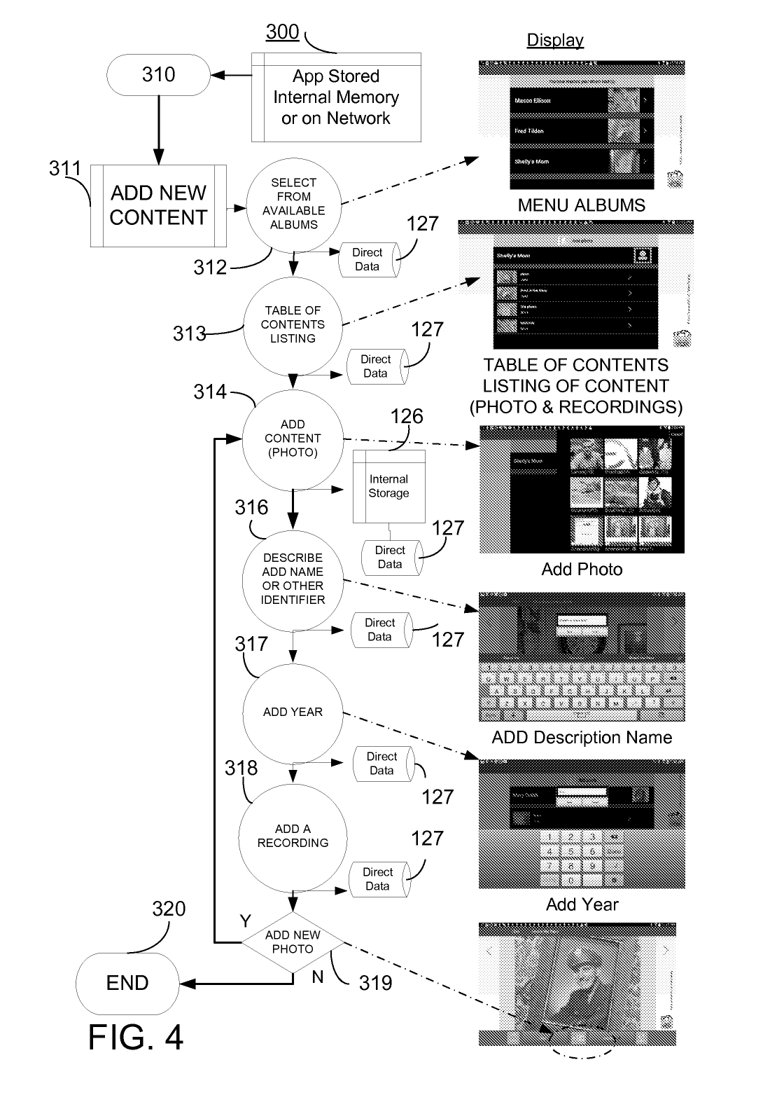

As is shown in FIG. 4, computer implemented process 300 generally adds a piece of content. As is shown in FIG. 5, computer implemented process 300, the process 340 adds additional audio recordings to a visual content stored. In Step 341, process starts to add additional audio recordings. The user selects from step 342 from available albums, and selects the visual content to add audio to in Step 343. In Step 344, Actual audio is added via the microphone 119 of the device 110 or by audio file. In step 345, the listing of contents is updated. In step 346, the user is prompted to add another recording to the same piece of content, if not, then the program ends at 347. As shown in FIG. 6, computer implemented process 300, the process 360 allows various playback functions and manipulation of the audio visual content. As is shown in FIG., computer implemented process 300, the process 340 allows for many administrative functions

In operation, the reminiscent therapy is accomplished on an individual and group basis. The device 110 is used to reflect on the person with dementia's life story using the memory triggers of the content (e.g. photographs, recordings, videos, writings, and other objects) The process of reflecting has been shown to have a positive benefit in the care and well-being of the person with dementia. The person with dementia can add such memory trigger content to the Tablet. Family members, carers and others in the reminiscent group therapy can also add memory trigger content to the Tablet. The Tablet 110 is used by the person with dementia to reflect on their life. The Tablet 110 is used by the person with dementia tell a story to others (i.e. family members, carers and others in the reminiscent group therapy) about the person with dementia's life. The benefits of this activity are that: the person hear's themselves talk, the person benefits from the memory triggers from the content about their life, and the Tablet 110 becomes a “mini joy machine” interface and the person resolves that they had a good life. The Tablet is used by the carers as a skilled nursing tool with the person with dementia to tell a story to others about the person with dementia's life. The system and computer implemented program on a Tablet provides reminiscent therapy treatment that has been shown to have benefits to help the person with dementia achieve a happy, comfortable state on a daily basis.

What is claimed is:

1. A method for reminiscence therapeutic application to improve quality of life of an individual with dementia, comprising the steps of:
(a) composing autobiographical memory components of the life of the individual with dementia in a portable computing device
(b) incorporating one or more visual elements in such autobiographical memory components, whereby said visual elements being configured to stimulate memory components of the life of the individual with dementia;
(c) incorporating one or more audio elements in such autobiographical memory components, whereby said visual elements being configured to stimulate memory components of the life of the individual with dementia;
(d) creating a composition of said one or more audio and/or visual elements of said autobiographical memory components of the life of the individual with dementia; and
(e) presenting said composition to the individual with dementia by playing back said one or more audio and/or displaying said one or more visual elements so as to stimulate improvement in the mood of the individual with dementia.

2. A method as recited in claim 1, wherein said step of presenting said composition to the individual with dementia further stimulates improvement by reducing the levels of depression and anxiety felt by the individual with dementia.

3. A method as recited in claim 1, wherein said step of presenting said composition involves the step of the individual with dementia presenting said composition to a group consisting essentially of persons, care-givers, carers, reminiscence group therapy members, nurses, health workers and the individual.

4. The method of claim 1, wherein said step of presenting said composition for playing on a schedule to the individual said one or more audio and/or displaying said one or more visual elements.

5. The method of claim 4, wherein said step of presenting said composition is according to a daily schedule lasting over a period of several months.

6. The method of claim 1, for therapeutically using said composition to treat a disorder in the individual with dementia further including the steps of:
(f) evaluating an individual's psycho-physiological state and composition to determine psycho-physiological needs and dysfunctions associated with the disorder;
(g) selecting playing back said one or more audio and/or displaying said one or more visual elements from said composition selected to treat the determined psycho-physiological needs and dysfunctions;
(h) using the device to play said composition to the individual so as to stimulate improvement in the mood of the individual; and
(i) repeating the step of using the device to play the composition to the individual according to a treatment schedule designed to treat the disorder.

7. A method as recited in claim 6, wherein the disorder is a disorder selected from the group consisting of:
depression;
anxiety;
dysfunctional psycho-physiological regulation capacity;
dysfunctional emotional self-regulation;
cognitive dysfunction;
hypertension;
psychiatric and neurological disorders;
neurodevelopmental disorders, and
pain.

8. A method as recited in claim 7, further comprising reevaluating the individual's psycho-physiological state and apparatus after repeating the step of using the audio device to play the audio program for a treatment time to determine results of the treatment.

9. An device for reminiscence therapeutic application to improve quality of life of an individual with dementia, said device comprising:
a readable medium for storing an computer program, wherein said computer program comprises: an input for receiving autobiographical memory components of the life of the individual with dementia, said autobiographical memory components comprising:
one or more visual elements in such autobiographical memory components configured to stimulate memory components of the life of the individual with dementia;
one or more audio elements in such autobiographical memory components configured to stimulate memory components of the life of the individual with dementia; and
a composition of said one or more audio and visual elements configured to constructively interact with the individual's autobiographical memory components;
a playback device for reading said readable medium, said play back device comprising:
a visual display output of said device for producing an visual output signal corresponding to said one or more visual elements of said composition; and
an audio output of said device for producing an audio output signal corresponding to said one or more audio elements of said composition.
